(19) United States
(12) Patent Application Publication
(10) Pub. No.: US 2009/0326369 A1
(43) Pub. Date: Dec. 31, 2009

(54) METHOD FOR ASCERTAINING THE POSITION OF A MEDICAL INSTRUMENT IN A BODY

(76) Inventors: Robert Schmidt, Munchen (DE); Alexander Urban, Markt, Schwaben (DE); Fritz Vollmer, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/490,435

(22) Filed: Jun. 24, 2009

Related U.S. Application Data
(60) Provisional application No. 61/094,894, filed on Sep. 6, 2008.

(30) Foreign Application Priority Data
Jun. 25, 2008 (EP) 08158987

(51) Int. Cl. A61B 5/05

(52) U.S. Cl. 600/424

(57) ABSTRACT

The present invention relates to a method for ascertaining the position of the head of a medical instrument in a vascular system, wherein the instrument can be inserted into the vascular system of a body and describes a path at least partially inside the vascular system, and the position of the instrument head is calculated from structural data, length data and the relative position between a reference point and the vascular system, wherein the instrument is guided past the reference point, the structural data represents the structure of the vascular system, and the length data represents the length of the path between the reference point and the instrument head.

### RELATED APPLICATION DATA

This application claims the priority of U.S. Provisional Application No. 61/094,894, filed on Sep. 6, 2008, which is hereby incorporated in its entirety by reference.

### FIELD OF THE INVENTION

The invention relates to: a method for ascertaining the position of a head of a medical instrument in a body, in particular in a vascular system; a program for performing the method; a storage medium on which the program is stored; a device for ascertaining the position of the head of a medical instrument in a body; and an operation system comprising a medical instrument and such a device.

### BACKGROUND OF THE INVENTION

In an operation or examination, medical instruments are often used which are inserted into the body to be operated on, in particular into a vascular system, wherein it is important to know the position of the head, i.e. the inserted end, of the medical instrument.

In previous practice, the position has been determined using real-time imaging by means of x-ray methods, wherein x-ray images are continuously produced. However, this procedure incurs a radiation load both for the patient and the physician.

Another approach, which is disclosed in the US patent application 2004/0171934 A1, is to use electromagnetic radiation generated at the head of the instrument. To this end, a number of coils are employed which firstly have to be integrated into the head of the instrument and secondly have to be supplied with energy by the instrument. This can necessitate a rather large instrument.

### SUMMARY OF THE INVENTION

It is therefore the object of the present invention to provide a method and device for ascertaining the position of the head of a medical instrument in a body, in which a radiation load can be reduced and in which in particular the overall size of the medical instrument does not have to be increased. The latter enables a broader range of application for the instrument.

This object is solved by the method, program, storage medium, device and operation system specified in the independent claims. Advantageous configurations may be gathered from the dependent patent claims.

One configuration of the invention relates to a method for ascertaining the position of the head of a medical instrument in the vascular system. The medical instrument can be inserted into a vascular system of a body. When the instrument is inserted, it describes a path at least partially inside the vascular system, wherein the instrument penetrates into the vascular system at an entry point and follows the course of the vascular system inside the body, wherein the course of the vascular system determines the path which the instrument takes inside the body. The position of the head of the instrument, referred to in the following as the instrument head, is situated inside the vascular system. The head is arranged at the inserted end of the instrument and for example occupies up to 1, 10 or 25 percent of the length of the instrument. If the instrument does not have a defined head, such as for example a flexible tube or a needle, then the inserted end is designated as the instrument head. The instrument is preferably flexible, for example a catheter or endoscope.

The position of the instrument head is calculated from: structural data which represents the structure of the vascular system; length data which represents the length of the path between a reference point—which the instrument is guided past—and the instrument head; and the relative position between the reference point and the vascular system. This relative position can for example be described by a position in a reference frame in which a model of the vascular system lies. The reference point preferably corresponds to the entry point of the instrument in the body, but can also be any other point, inside or outside the body, which lies on the path of the instrument. The path reflects the shape of the instrument in its extension in the longitudinal direction, for example the bend in a flexible tube-shaped instrument.

The length data accordingly describes the length of the part of the instrument between the instrument head and the reference point. The reference point advantageously lies inside the vascular system or at the entry point of the instrument into the vascular system. The structural data contains the three-dimensional configuration of the vascular system and are preferably ascertained before the instrument is inserted into the vascular system, for example as an angiogram using x-ray irradiation, magnetic resonance tomography or computer tomography. The relative position between the reference point and the vascular system establishes a three-dimensional relationship by rendering the position of the reference point relative to the vascular system. It is either known beforehand or is ascertained and/or measured. One way is to mark the reference point in the structural data manually, for example before, during or after the instrument is inserted. Another way is to measure the position of the body (and therefore the vascular system) and the reference point with the aid of marker devices, such as are described below.

If the relative position between the reference point and the vascular system, the structure of the vascular system (for example as a model) and the length of the path between the reference point and the instrument head are known, then a finite number of positions at which the instrument head can be situated exist inside the vascular system. The actual position is dependent on the path which the instrument describes in the vascular system. This path is usually planned before the instrument is inserted into the body, such that this results in the position—of the number of possible positions—which corresponds to the actual position of the instrument head in the vascular system. The calculated position of the instrument head can be used by the physician, in particular when navigating the instrument. The physician can in particular use the calculated position during the inserting process, in order to check whether the planned path matches the actual path which currently obtains. How the calculated position can be used to this end is illustrated further below.

Branch data, which represents the branches which the instrument has followed in the vascular system, is preferably additionally incorporated into calculating the position of the instrument head. The branch data is for example stored in a table which contains the path length at which branching occurs, and in which direction. A second way is to mark the branches in the structural data. If the branch data is taken into account in the calculation, then it is for example possible to identify, from the calculated position of the instrument head, whether the instrument is following the path planned beforehand. The branch data can for example be ascertained on video images recorded inside the vascular system, in the region of the instrument head, while the instrument is being inserted. Within the framework of this document, the term “video image” means both static images and moving images. Alternatively, the branch data can be obtained from ultrasound images, x-ray images, magnetic resonance tomography recordings and computer tomography recordings, or output signals of a gyro sensor on the instrument head.

In a preferred embodiment of the invention, the length data is calculated from a relative movement between the instrument and the reference point, wherein the term “relative movement” refers to the entirety of a distance traveled and a movement direction. If the instrument has moved relative to the reference point by a certain path length, then the instrument head has moved by precisely this path length, deeper into the vascular system or out of the vascular system, wherein the relative movement need not necessarily be measured directly at the reference point itself, but can also be measured at another point, i.e. indirectly. If, for example, the reference point is situated inside the vascular system, the relative movement can also then be ascertained between the instrument and for example the entry point of the instrument into the body, and equated with the relative movement between the instrument and the reference point, for it may be assumed that the relative movement between the instrument and the entry point is equal to the relative movement between the instrument and any other position along the path, i.e. also the reference point.

The relative movement is preferably ascertained by optical measurement, in particular from a marking on the instrument. The marking can for example be a barcode, a color code or a reflective marking. An absolute length value can be encoded into the marking. The marking can also be a regular pattern, the relative movement of which in relation to the reference point is accumulated to give the length of the path. A combination of image detection and image processing is also possible, such as is for example performed in the case of an optical computer mouse.

As an alternative to or in addition to optical measurement, the relative movement can also be ascertained by a mechanical measurement, for example by means of a scroll wheel which rolls off on the instrument.

A second configuration of the present invention relates to a method for ascertaining the position of a head of a medical instrument which can be inserted into a body and which describes a path at least partially inside the body. As compared to the first configuration, the path of the instrument is not limited to following the structure of a vascular system.

The position of the instrument head is calculated from: course data which represents the course of the path; length data which represents the length of the path between a reference point—which the instrument is guided past—and the instrument head; and the relative position between the reference point and the body, wherein a relative movement between the instrument and the reference point is automatically ascertained, and the length data is automatically calculated from the relative movement.

This configuration of the invention is particularly suitable for rigid medical instruments such as for example biopsy needles or screws. The path of a rigid medical instrument corresponds to a straight line and is therefore known.

The designs described in the following can be applied to both the first and second embodiment.

The position of the entry point—and therefore a possible reference point relative to the body and/or vascular system—can be determined on the basis of a marker device, for example a marker star, or individual marker elements (arranged stationary with respect to each other). A marker star is an object comprising three or more spatially arranged spheres which are fixedly connected to each other. The spatial positions of the spheres can be detected for example by means of a 3D camera. Since the positions of the spheres relative to each other are known, both the position and the orientation of the marker star and therefore the position of an object marked using the marker star can be ascertained from the positions of the spheres.

Such an object is for example a device for measuring the relative position between the reference point and the vascular system and/or body. This device is preferably placed on the body at the entry point of the instrument. In particular, the relative position between the marker device and the reference point is known, such that by detecting the marker device, it is possible to determine the position of the reference point, in particular relative to the path.

In one configuration of the invention, a signal is generated when the calculated position of the instrument head corresponds to a predetermined position. The predetermined position is for example the desired end position of the instrument head, or a (possible) branch in the vascular system. The signal is for example an indication signal or warning signal to the physician, or a rinsing signal which triggers the introduction of a rinsing liquid by the instrument, in order to enable a video image to be recorded in the region of the instrument head.

In another development of the present invention, the actual (current) position of the instrument head is ascertained. This is for example achieved by means of imaging methods such as x-raying or computer tomography or electromagnetically determining the position by means of coils with current flowing through them, the field from which is detected. The actual position is the position which the instrument head is currently occupying in reality. The actual position is for example ascertained when the calculated position corresponds to a critical position, for example the desired end position of the instrument head or a branch in the vascular system. Since the actual position is not ascertained while the instrument is being inserted, or only rarely and at greater intervals in time than when ascertaining the position in a purely x-ray-based way, the radiation load both for the patient and for the physician is significantly reduced, even when an x-ray method is used.

In a preferred embodiment of the invention, the reference point and/or the length data are adapted with the aid of the actual position of the instrument head. This is for example achieved by setting the actual position of the instrument head as the reference point and setting the path length (traveled by the instrument head) in the length data to zero. This creates a new starting point for the method in accordance with the invention, on which determining the position is subsequently based.

Alternatively or additionally, the length data can be modified. One way is to replace the length data with theoretical length data. The theoretical length data is the length data which would result in a match between the calculated position and the actual position if the calculation were based on it. This theoretical length data is then used as a basis when the instrument subsequently moves, for example when the length data is updated on the basis of a relative movement between the reference point and the vascular system and/or body. A second way is to calculate a scaling factor, by which the path length is multiplied. The scaling factor is determined such that the calculated position would correspond to the actual position if the calculation were based on the path length multiplied by the scaling factor. The path length multiplied by the scaling factor is incorporated into the length data when the position is subsequently calculated.

The invention also relates to a program which, when it is loaded onto a data processing device or is running on a data processing device, causes the data processing device to perform the method described above. The invention also relates to a storage medium, on which such a program is stored, or to a signal wave which carries information constituting such a program.

The invention also relates to a device for ascertaining the position of the head of a medical instrument, comprising a computer on which the program described above is running or is loaded.

The invention also relates to an operation system comprising a medical instrument and a device as described above. In one configuration of the invention, the operation system comprises a camera in the region of the instrument head for capturing a video image. The video image shows an interior view of the body and/or vascular system and can be used both to detect the actual position of the instrument head and when navigating the instrument, for example at branches of the vascular system. If, for example, the video image shows a branch in the vascular system, but a branch is not present at the calculated position of the instrument head, then the path of the instrument does not correspond to the desired path. Given the path length and the information that the instrument head is situated at a branch, it is possible to ascertain a number of paths which describe the instrument in the vascular system. From this number, the physician can determine the actual (current) path of the instrument, for example on the basis of the size and/or structure of the vessels in front of and/or behind the branch.

A deviation between the actual position and the desired path also exists when the video image does not show a branch, but the calculated position of the instrument head is situated at a branch.

In another embodiment, the operation system comprises a device for determining the actual position of the instrument head. This device is for example an x-ray apparatus, a computer tomograph or an electromagnetic position ascertaining system.

The operation system preferably comprises a camera which is directed onto the body, for capturing an image from which the length data can be ascertained. The camera is for example an infrared camera. An infrared camera has the advantageous that its sensitivity lies in the non-visible frequency spectrum, and there is no additional illumination in this frequency range to distract the physician and the patient.

The length data can be ascertained from the image from the camera for example by detecting a marking in the form of a barcode, a color code or a reflective marking on the instrument, which in particular represents a path length traveled. A marking can also be a marker star such as has already been described above. The marker is then arranged on a part of the instrument which is not inserted into the body. The camera is accordingly situated outside the body and detects the marking on the instrument. A marker can also be provided as a reference for marking the entry point. It is then also for example possible to detect a torsion between the instrument and the entry point.

### BRIEF DESCRIPTION OF THE DRAWINGS

The present invention shall be illustrated in more detail on the basis of an example embodiment.

FIG. 1 shows an operation system in accordance with the invention.

FIG. 2 shows an endoscope in a vascular system.

FIG. 3 shows a biopsy needle.

### DETAILED DESCRIPTION

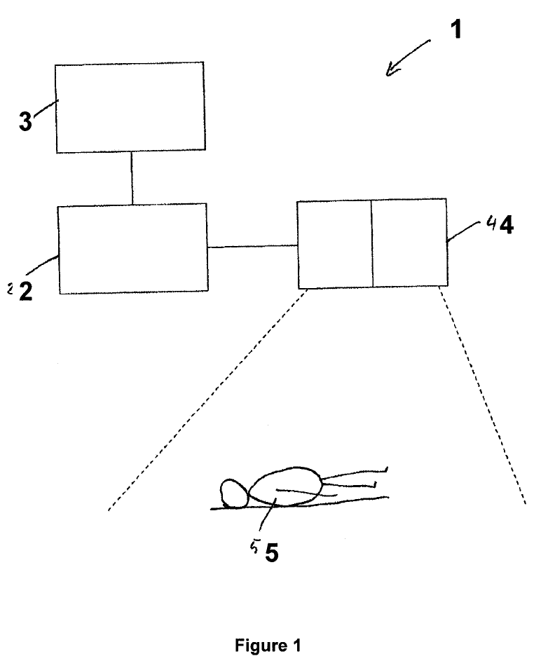

FIG. 1 schematically shows an operation system 1 comprising a computational unit 2, a monitor 3 and a 3D infrared camera 4. A patient 5 to be examined is situated within the detection range of the camera 4 shown by broken lines.

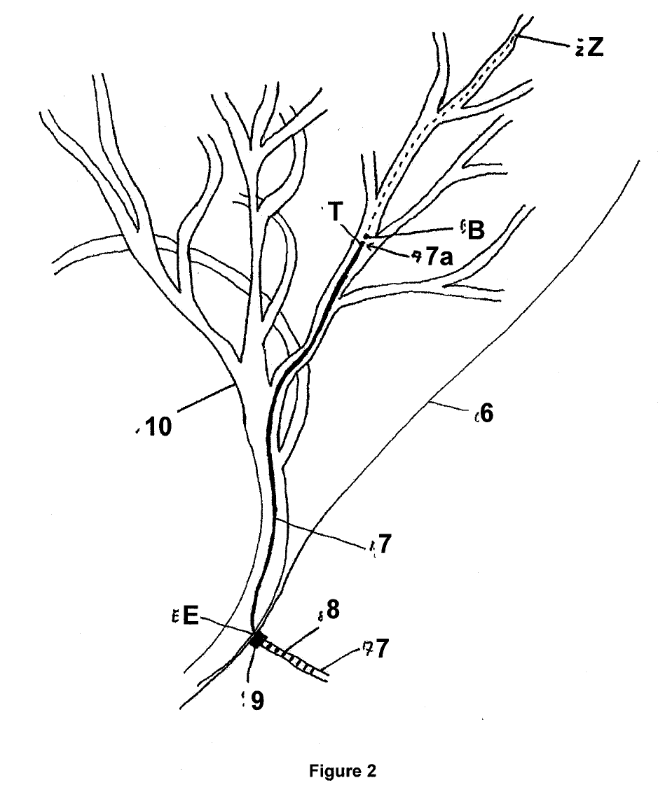

FIG. 2 shows a part of the vascular system 10 of the patient 5 which is situated below the skin 6. The vascular system 10 is to be examined at a position Z by means of an endoscope 7. The endoscope 7 is inserted into the vascular system 10, which lies directly below the skin 6, at an entry point E. The structure, i.e. the three-dimensional configuration, of the vascular system 10 has been ascertained, by means of a computer tomograph, before the endoscope 7 is inserted. The planned path between the entry point E and the target point Z is shown by a broken line.

A regular marking 8 is situated on the endoscope 7, in the form of equidistant bars which are detected by a measuring device 9 at the entry point E. Two light sources and two photodetectors are arranged in the measuring device 9, using which it is possible to ascertain the direction in which and the path length by which the endoscope 7 has been inserted into or drawn out of the vascular system 10 relative to the measuring device 9 and/or entry point E. To this end, the measuring device 9 is connected to the computational unit 2.

The position of points, such as the reference point or the entry point E, and the position and/or orientation of objects, such as the body 5 or the measuring device 9, can be measured using marker devices or a pointer, or directly input into the computational unit 2. A pointer is a device comprising a marker device. The pointer is held at a point to be detected, and the position of the point is calculated from the position of the marker device. The position of the marker device is ascertained for example with the aid of the camera 4.

The entry point E initially serves as a reference point for determining the position of the head 7a of the endoscope 7. The position of the entry point E as compared to the patient 5 and therefore the vascular system 10 is known to the computational unit 2, for example from measurements. The position and/or orientation of the body 5 is determined for example from the positions of characteristic points of the body 5, so-called landmarks. Alternatively, a marker star is situated on the body 5. At the point in time shown in FIG. 2, the instrument head 7a has been inserted into the vascular system 10 as far as the position T. On the path between the entry point E and the position T, the endoscope 7 has followed the branches left, right and left in the vascular system 10.

From the markings 8 on the endoscope 7, the measuring device 9 and the computational unit 2 have calculated length data which represents the length of the path between the entry point E and the position T of the instrument head 7a. The computational unit 2 calculates the position of the instrument head 7a inside the vascular system 10 from said length data, together with the relative position between the reference point E and the vascular system 10, and from structural data which represents the structure of the vascular system 10. This position usually matches the actual position of the instrument head to a sufficient level of accuracy.

FIG. 2 shows a scenario in which the calculated position B does not exactly match the actual position T, since the path of the endoscope 7 in the vascular system 10 does not always take the shortest possible route. The actual position T is ascertained in a check measurement by means of an x-ray apparatus (not shown) and compared by the computational unit 2 with the calculated position B. On the basis of the deviations between the two positions, the actual position T is defined as a new reference point. When subsequently calculating the position of the instrument head 7a, this new reference point T and the path length traveled by the relative movement between the endoscope 7 and the new reference point are assumed beyond the position T. Since the traveled relative movement cannot be directly measured at the position T, the measurement is again taken at the entry point E. In subsequent calculations, it is thus assumed that the relative movement between the endoscope 7 and the entry point E corresponds to the relative movement between the endoscope 7 and the new reference point T.

If calculating the position reveals that the instrument head 7a is situated just short of the target position Z, then the computational unit 2 for example generates a warning signal for the physician and/or a rinsing signal which causes the operation system 1 to inject a rinsing liquid into the vascular system 10, in order to enable a video image to be recorded by a camera situated on the instrument head 7a.

Instead of an endoscope 7, the instrument can also be another flexible medical instrument, for example a catheter. In contrast to the position ascertaining methods from the prior art, the method in accordance with the invention exhibits the advantage that the position of the medical instrument is not continuously determined using x-ray recordings, therefore exposing both the physician and the patient to a radiation load, but rather an x-ray recording is only optionally used to verify the calculated position of the instrument head.

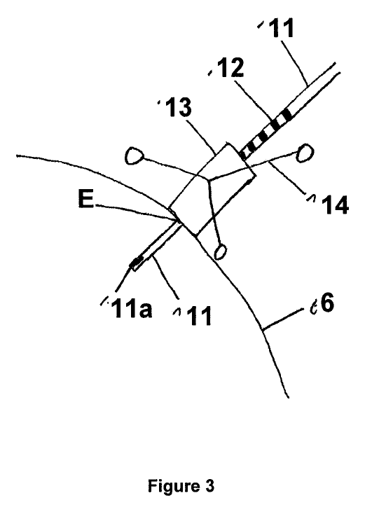

In the example application according to FIG. 3, a biopsy needle 11 comprising a sample tip 11a has been inserted into the body of the patient 5. To this end, an inserting device 13—a so-called guiding tube—has been placed onto the skin 6 of the patient 5 at the entry point E. The biopsy needle 11 can be guided linearly through the inserting device 13. Optionally, a marker star 14 is fixedly connected to the inserting device 13 and comprises three spatially arranged spheres. The camera 4 detects the spatial positions of the spheres. On the basis of the known arrangement of the spheres, it is possible to unambiguously calculate the position and alignment of the inserting device 13 relative to the body of the patient 5 from the 3D image from the camera 4, if the position and orientation of the body 5 are also known. The latter are determined for example with the aid of a marker star or the position of landmarks.

When the rigid biopsy needle 11 is inserted into the body, the testing tip 11a describes a linear path. The length of the path, i.e. the penetration depth of the testing tip 11a relative to the skin 6, is ascertained by the inserting device 13 on the basis of a barcode 12 on the biopsy needle 11. The path length is calculated in the same way as in the example application according to FIG. 2, by means of two light sources and two photodetectors.

In an alternative to the embodiment shown, the relative movement between the reference point E and the biopsy needle 11 is not determined in the inserting device 13 but rather from the image from the camera 4 which detects the barcode 12 on the biopsy needle 11.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiments.

Although the invention has been shown and described with respect to one or more particular preferred embodiments, it is clear that equivalent amendments or modifications will occur to the person skilled in the art when reading and interpreting the text and enclosed drawings of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment or embodiments illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A method for ascertaining the position of the head of a medical instrument in a vascular system, wherein the instrument can be inserted into the vascular system of a body and describes a path at least partially inside the vascular system, and the position of the instrument head is calculated from structural data, length data and the relative position between a reference point and the vascular system, wherein the instrument is guided past the reference point, the structural data represents the structure of the vascular system, and the length data represents the length of the path between the reference point and the instrument head.

2. The method according to claim 1, wherein branch data, which represents the branches which the instrument has followed in the vascular system while inserted, is additionally incorporated into calculating the position of the instrument head.

3. The method according to claim 1, wherein the length data is calculated from a relative movement between the instrument and the reference point.

4. The method according to claim 3, wherein the relative movement is ascertained by optical measurement.

5. The method according to claim 4, wherein the relative movement is ascertained from a marking on the instrument.

6. A method for ascertaining the position of the head of a medical instrument in a body, wherein the instrument can be inserted into the body and describes a path at least partially inside the body, and the position of the instrument head is calculated from course data, length data and the relative position between a reference point and the body, wherein the instrument is guided past the reference point, a relative movement between the instrument and the reference point is automatically ascertained, the length data is automatically calculated from the relative movement, the course data represents the course of the path, and the length data represents the length of the path between a reference point and the instrument head.

7. The method according to claim 1, wherein a signal is generated when the calculated position of the instrument head corresponds to a predetermined position.

8. The method according to claim 1, wherein the actual position of the instrument head is ascertained.

9. The method according to claim 8, wherein the reference point and/or the length data are adapted from the actual position of the instrument head.

10. A program which, when it is loaded onto a data processing device or is running on a data processing device, causes the data processing device to perform the method according to claim 1.

11. A storage medium, on which the program according to claim 10 is stored, or a signal wave which carries information constituting the program according to claim 10.

12. A device for ascertaining the position of the head of a medical instrument, comprising a computer on which the program according to claim 10 is running or is loaded.

13. An operation system comprising a medical instrument and a device according to claim 12.

14. The operation system according to claim 13, comprising a camera in the region of the instrument head for capturing a video image.

15. The operation system according to claim 13, comprising a device for determining the actual position of the instrument head.

16. The operation system according to claim 13, comprising a camera which is directed onto the body, for capturing an image from which the length data can be ascertained.
